(19) United States
(12) Patent Application Publication
(10) Pub. No.: US 2006/0148832 A1
(43) Pub. Date: Jul. 6, 2006

(54) Use of 2-amino-2h-quinazoline derivatives for producing therapeutic agents

(76) Inventor: Rolf Sachse, Berlin (DE)

Correspondence Address: Kriegsman & Kriegsman, 665 Franklin Street, Framingham, MA, 01702, US

(21) Appl. No.: 10/541,535

(22) PCT Filed: Jan. 7, 2004
(86) PCT No.: PCT/EP04/00054

§ 371 Date: Jul. 9, 2005

(30) Foreign Application Priority Data
Jan. 9, 2003 (DE) 103-01-105.6

(51) Int. Cl. A61K 31/517

(52) U.S. Cl. 514/266.4

(57) ABSTRACT

The invention concerns the use of 2-amino-2H-quinazoline derivatives of general chemical formula (I), wherein R1 indicates an alkyl group with 1-5 carbon atoms and R2, R3, R4 and R5, independently of one another, each indicate a chlorine or hydrogen atom, as well as their pharmaceutically compatible salts, for producing therapeutic agents for the treatment of myeloproliferative diseases, high blood pressure and for bronchodilation.

The present invention concerns the use of 2-amino-2H-quinazoline derivatives as well as their pharmaceutically compatible salts for producing therapeutic agents for the treatment of myeloproliferative diseases, high blood pressure and for bronchodilation.

Myeloproliferative diseases belong to diseases that concern the bone marrow. The common feature of myeloproliferative diseases is an independent propagation of all blood cells of the bone marrow (red and white blood cells as well as platelets). These diseases include, among others, polycythemia vera and essential thrombocythemia. With increasing progression, the former can be accompanied by thrombocytoses.

Thrombocythemia is a malignant disease of the bone marrow, which is characterized by an increased number of platelets (thrombocytes) in the blood. The cause of thrombocythemia is a defect of the stem cells of the hematopoietic system, which is the origin of all blood cells and is formed in the bone marrow. The large precursor cells of thrombocytes are megakaryocytes, which are formed to a great extent in thrombocythemia. When a single megakaryocyte bursts, several thousand platelets without nuclei are released. Therefore, in the treatment of myeloproliferative diseases, such as essential thrombocythemia, in addition to other therapies, the compound 6,7-dichloro-1,5-dihydroimidazo-[2,1-b]-quinazolin-2-[3H]-one is used, better known under the name anagrelide (utilized preferably as the hydrochloride) (e.g.: Anagrelide: a new drug for treating thrombocytosis (Silverstein, M. N. et al. N Engl J Med (1988) 318(20): 1292-4). Anagrelide is a substance, which is characterized by a selective thrombocyte-reducing effect, for example, described by Lindley et al. (Anagrelide: a novel agent for the treatment of myeloproliferative disorders. Pescatore S L, Lindley C, Expert Opin Pharmacother (2000) 1(3): 537-46).

A dose of 3 mg/day, for example, after one week, causes a decrease in thrombocytes of approximately 50 percent, indicating that anagrelide intervenes—via a still unknown inhibiting effect—in megakaryocytopoiesis.

Other effects of anagrelide are described in U.S. Pat. No. 3,932,407 and U.S. Pat. No. 4,146,718, e.g., for use as an anti-aggregative agent for blood platelets or an agent that decreases blood pressure as well as for the treatment of bronchodilation. Ethyl-(2-amino-5,6-dichloro-2H-quinazolin-3-yl) acetate hydrobromide is also mentioned in U.S. Pat. No. 4,146,718 as having an anti-aggregative effect on blood platelets.

The compound 6,7-dichloro-1,5-dihydroimidazo-[2,1-b]-quinazolin-2-[3H]-one and its production were described for the first time in U.S. Pat. No. 3,932,407, wherein a synthese route is described with the use of phosphorus oxychloride via an anagrelide precursor, e.g., a 2-chloro-2H-quinazoline derivative, which is then cyclized to the anagrelide base, a compound that is difficultly soluble in water. In order to improve water solubility, such active substances are converted to salts, since frequently they can be absorbed by the body only in this form. Since then, the synthesis path used for anagrelide has been modified relative to specific intermediates in several other publications.

A synthesis route is described in: “The Organic Chemistry of Drug Synthesis” (D. Lednicer and L. A. Mitscher, Wiley-lnterscience; 1st Edition (Nov. 29, 1984) Vol. 3, p. 244, ISBN: 0471092509), which starts with a 2,3-dichloro-6-nitrobenzyl chloride compound, in contrast to U.S. Pat. No. 3,932,407. After alkylation, the reduction of the nitro group leads to an aniline derivative. This is converted to a cyanamide, which is then converted to the quinazoline with the release of hydrocyanic acid and further to the anagrelide.

In U.S. Pat. No. 4,146,718 and U.S. Pat. No. 5,801,245, the prior use of phosphorus oxychloride, which is objectionable for health reasons, is avoided by the preparation of a 2-amino-2H-quinazoline derivative, which is then converted in anhydrous ethanol with an organic base to the anagrelide base by boiling under reflux for several hours.

U.S. Pat. No. 5,391,737 describes a thermal cyclization in a hydrochloric acid medium, which forms the anagrelide base via a cyano-containing quinazoline derivative.

An alternative synthesis path for the production of anagrelide via 2,3-dichlorobenzaldehyde as the initial substance is disclosed in U.S. Pat. No. 6,388,073 B1. This alternative synthesis route also leads to the known anagrelide precursor of a 2-amino-2H-quinazoline derivative, which is then cyclized at room temperature in aqueous medium with the use of an organic base.

It is common to all the methods employed that the anagrelide base is purified after it is obtained and then must be converted into a pharmaceutically compatible salt. Organic bases, such as triethylamine or pyridine, must be completely eliminated, since these are compounds that are very damaging to health. The conversion to the salt is also a very sensitive step. Depending on the system, it can induce acid hydrolysis of the lactam ring of the anagrelide, which is viewed as a disadvantage. This hydrolysis also occurs after the product is dried, which negatively influences the long-term stability of the active substance.

The incidence of polycythemia vera or essential thrombocythemia as a disease [in the population] is 0.5-1 person per 100,000 individuals and per year. Polycythemia vera and essential thrombocythemia belong to disorders that occur relatively rarely and are thus also called “orphan diseases”. Based on the limited number of potential patients, the development of drugs specifically effective for orphan diseases is hardly considered economical. Such drugs are characterized by high development costs with a simultaneous small market. The production costs of anagrelide, which are also high, are frequently based on a large number of synthesis intermediates, which are produced with rather variable small yields, whereby the yields are further reduced by the purification steps. The long-term stability of anagrelide is also negatively influenced by the necessity of producing this compound as a pharmaceutically compatible hydrate/hydrochloride salt, due to the low solubility in water of the anagrelide base, so that there is a slowly progressing hydrolysis of the lactam ring due to the hydrochloric acid and the hydrate water contained therein.

The object that is the basis of the invention thus consists of preparing a new active substance for the treatment of myeloproliferative disorders, high blood pressure and for bronchodilation, wherein the active substance shall be produced in a simpler manner, of moderate cost and compatible with the requirements for environmental compatibility as well as compatible with the production conditions. The problem of the limited storage stability of anagrelide due to hydrolysis will also be avoided.

The object will be solved by the use of 2-amino-2H-quinazoline derivatives of the general formula I and their pharmaceutically compatible salts for producing therapeutic agents for the treatment of myeloproliferative diseases, high blood pressure and for bronchodilation according to claim 1.

Preferred embodiments of the invention are given in the subclaims.

The 2-amino-2H-quinazoline derivatives according to the invention have the following general chemical formula I.

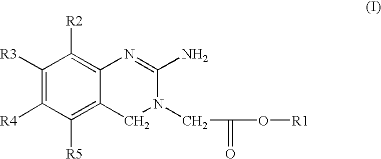

Therein, R1 is an alkyl group with 1-5 carbon atoms and R2, R3, R4 and R5, independently of one another, each represent a chlorine or hydrogen atom. The alkyl groups can be straight-chain or branched alkyl groups with 1-5 carbon atoms, comprising methyl, ethyl, n-propyl, iso-propyl, n-butyl, sec-butyl, tert-butyl, n-pentyl, sec-pentyl, tert-pentyl and neo-pentyl. Preferred are compounds in which R1 is methyl or ethyl. Further preferred are compounds in which at least two residues of R2, R3, R4 and R5 are chlorine atoms. Particularly preferred are compounds in which R1 stands for a methyl or ethyl, R2 and R3 are each hydrogens, and R4 and R5 are each chlorine atoms.

The present invention also comprises pharmaceutically compatible salts of the compound of formula I for application particularly in the treatment of myeloproliferative diseases, high blood pressure and for bronchodilation. Suitable pharmaceutically compatible salts of the compound of formula I are acid addition salts with organic and inorganic acids, such as, e.g., the hydrochloride, hydrobromide, sulfate, fumarate, maleate, lactate and succinate.

Preferred 2-amino-2H-quinazoline derivatives for solving the object [of the invention] are methyl-(2-amino-5,6-dichloro-3,4-dihydro-2H-quinazolin-3-yl) acetate hydrochloride hemihydrate and ethyl-(2-amino-5,6-dichloro-3,4-dihydro-2H-quinazolin-3-yl) acetate hydrochloride hemihydrate as well as methyl-(2-amino-5,6-dichloro-3,4-dihydro-2H-quinazolin-3-yl) acetate hydrobromide hemihydrate and ethyl-(2-amino-5,6-dichloro-3,4-dihydro-2H-quinazolin-3-yl) acetate hydrobromide hemihydrate. The contained water of crystallization may also be present in other molar ratios or, optionally, it may also be absent.

The synthesis of the initial compounds of general formula I can be conducted, for example, by the synthesis route of the above publications U.S. Pat. No. 4,146,718 or U.S. Pat. No. 6,388,073.

A synthesis route is preferably used, which is taken from the synthesis route in the above-named handbook, “The Organic Chemistry of Drug Synthesis” (D. Lednicer and L. A. Mitscher, Wiley-Interscience; 1st Edition (Nov. 29, 1984) Vol. 3, p. 244).

The salts can be produced in a known way by addition of the corresponding acid to a methanolic slurry of a 2-amino-2H-quinazoline derivative of formula I. Formation of a salt is also conceivable in the stomach of mammals, analogous to the salt formation of anagrelide in U.S. Pat. No. 6,388,073.

The 2-amino-2H-quinazoline derivatives according to the invention can be cyclized to the anagrelide in aqueous medium as a function of the pH without addition of other adjuvants or boiling under reflux for hours. By cyclization in aqueous, slightly alkaline solution, the derivatives according to the invention of general formula I, wherein R1 is an alkyl group with 1-5 carbon atoms and R2, R3, R4 and R5, independently of one another, each indicate a chlorine or hydrogen atom, can be directly applied without further synthesis expenditure as so-called prodrug compounds for application, particularly in the case of myeloproliferative diseases and high blood pressure in mammals and particularly in humans, since it has been demonstrated that the prevailing pH in mammals, particularly in the intestine, of 8-10 leads to an in vivo cyclization of this compound and thus to the formation of the active substance, anagrelide. A cyclization of the compound with the general chemical formula I in strongly acidic pH range, as prevails, for example, in the stomach of mammals, has only been detected up to a certain small percentage.

If the term “prodrug” is used, it refers to a compound of general formula I or to its pharmaceutically compatible salts, since these salts represent a direct precursor to the active substance, anagrelide, and under the given circumstances, by cyclization, lead to the actual active substance, anagrelide, known from the prior art or an anagrelide derivative.

The described use of organic bases that are damaging to health, such as, e.g., triethylamine or pyridine, for the cyclization of the compound according to the invention is omitted in the production of anagrelide. This result leads to a savings with regard to further synthesis steps for the production of anagrelide and anagrelide salts and their subsequent downstream purification and thus there is a higher production yield. In addition, the problem of hydrolysis of the active substance is excluded when it is in salt form.

The therapeutic agent is a pharmaceutical preparation, which contains at least one compound of general formula I as the active substance, and optionally other active substances, as well as at least one pharmaceutical adjuvant. Preferably, these agents are used for the treatment of myeloproliferative diseases, such as essential thrombocythemia and polycythemia vera (in conjunction with thrombocytosis). In addition, the preparation according to the invention can be employed for the treatment of high blood pressure and for bronchodilation. Corresponding pharmaceutical formulations are prepared or produced in a way known in and of itself, from at least one compound of general formula I and/or its pharmaceutically compatible salts and pharmaceutically compatible adjuvants, particularly support materials, solvents, fillers, diluents, colorants and/or binding agents. The selection of the solid or liquid adjuvants that are utilized, as well as their quantities, depend on how the pharmaceutical is administered.

Preferred preparations consist of a form of administration, which is suitable for oral, enteral, intravenous (i. v.) or local application. Such forms of administration are, for example, tablets, film tablets, dragees, pills, capsules, powders, liquids such as syrups, gels, injectable liquids for intravenous injection, etc. In addition, slow-release forms, such as implantable preparations as well as suppositories, are suitable. In this way, the individual preparations release the derivatives according to the invention in the body gradually or they release the total quantity in a short time, depending on which type is used each time.

For oral administration, capsules, pills, tablets, dragees and liquids or other known oral forms of administration can be utilized as pharmaceutical preparations for oral administration. In this case, the pharmaceuticals can be formulated in such a way that they release the active substances either in a short time and provide them to the body, or have a slow-release effect, so that a longer-lasting, slow introduction of the active substance is achieved in the body. In addition to the at least one 2-amino-2H-quinazoline derivative and/or its pharmaceutically compatible salts, the dosage units may contain one or more pharmaceutically compatible adjuvants, for example, substances for adjusting the rheology of the pharmaceutical, surface-active agents, solubilizers, microcapsules, microparticles, granulates, diluents, binding agents, such as starch, sugar, sorbitol and gelatins, and also fillers, such as silicic acid and talcum, lubricants, colorants, fragrances and other substances.

Corresponding tablets can be obtained, for example, by mixing the active substance with known adjuvants, for example, inert diluents such as dextrose, sugar, sorbitol, mannitol, polyvinylpyrrolidone, bursting agents such as corn starch or alginic acid, binding agents such as starch or gelatins, lubricants such as carboxypolymethylene, carboxymethylcellulose, cellulose acetate phthalate or polyvinyl acetate. The tablets may also consist of several layers.

Dragees can be produced correspondingly by coating of cores produced analogously to the tablets with agents commonly used in dragee coatings, for example, polyvinylpyrrolidone or shellac, gum arabic, talc, titanium oxide or sugar. The dragee envelope may also consist of several layers, wherein the adjuvants mentioned above in the case of tablets can be used.

Capsules containing active substances can be produced, for example, by mixing the active substance with an inert carrier such as milk sugar or sorbitol and encapsulating this mixture in gelatin capsules.

The 2-amino-2H-quinazoline derivatives according to the invention may also be formulated in the form of a solution, which is specific for oral administration and which contains as components, in addition to the active derivative, at least one pharmaceutically compatible oil and/or at least one pharmaceutically compatible lipophilic, surface-active substance and/or at least one pharmaceutically compatible hydrophilic, surface-active substance and/or at least one pharmaceutically compatible water-miscible solvent.

The substances according to the invention may also be applied in suitable solutions such as, for example, physiological saline solution, as an infusion solution or injection solution. For parenteral application, the active substances may be dissolved or suspended in at least one physiologically compatible diluent. Solubilizers can be added to increase the solubility.

In order to formulate an injectable preparation, any liquid vehicle can be used, in which the compounds according to the invention are dissolved or emulsified. These liquids also frequently contain substances for regulating viscosity, surface-active substances, preservatives, solubilizers, diluents and other additives, with which the solution will be adjusted isotonically. Other active substances may also be administered together with the derivatives according to the invention.

The substances according to the invention may also contain adjuvants, which react basically in aqueous solutions, so that the cyclization of the substances according to the invention can be produced, for example, in an inhalation bath or in a spray solution for bronchodilation or in a slow-release form inside or outside the body of the mammal.

The dosage of the derivatives of general formula I according to the invention is determined by the treating physician and depends, among other things, on the weight of the patient, the indication, the type of application and the severity of the disorder. The daily dosage amounts to no more than 5 mg, usually 0.5 to 5.0 mg, wherein the dose can be given once as a single dose to be administered or it can be divided into two or more daily doses.

The following examples serve to explain the invention, but do not limit the general concepts of the invention, particularly not the use of various optionally substituted compounds of the general formula I. The yields of the compounds produced are not optimized. The temperatures are not corrected.

In the following examples, the ethyl and methyl (2-amino-5,6-dichloro-2H-quinazolin-3-yl) acetate derivatives employed were produced each time beginning with 2,3-dichloro-6-nitrobenzyl chloride by means of the following three-step synthesis:

A) Ethyl (2-amino-5,6dichloro-2H-quinazolin-3-yl) acetate hydrobromide (MW 383.1)

First step—N-(2,3-dichloro-6-nitrobenzyl) glycine ethyl ester hydrochloride (MW 343.6)

As the reaction mixture, 818.5 g of 2,3-dichloro-6-nitrobenzyl chloride in 3700 ml of acetonitrile were placed in a 10-liter reaction flask while stirring and 724.5 g of glycerin ethyl ester hydrochloride were added. Then 2415 ml of triethylamine were dripped into this reaction mixture while stirring within 30-50 min. Next, the reaction mixture was boiled under reflux at approximately 75° C. for 2 h and the conversion was followed by means of HPLC. It was then cooled to room temperature.

In order to obtain the reaction product, the liquid phase was separated from the precipitated triethylamine*HCl, whereby the precipitated triethylamine*HCl was washed with approximately 1800 ml of acetonitrile. Washing phase and liquid phase were combined and concentrated to dryness on the rotary evaporator. The residue was washed with tetrahydrofuran (THF) at approximately 5° C. The liquid phase was separated from the precipitated triethylamine*HCl, the precipitated triethylamine*HCl was washed with THF and the combined organic phases were concentrated to dryness on the rotary evaporator. The thus-obtained reaction product was dissolved in triple the weight quantity of ethanol while stirring at room temperature and reacted with 5-6 N hydrochloric acid (in 2-propanol). The batch was cooled to approximately 5° C., the precipitated crude product was aspirated, washed with ethanol and dried in the drying cabinet at approximately 60° C. The yield of the reaction product N-(2,3-dichloro-6-nitrobenzyl) glycine ethyl ester hydrochloride amounted to 911 g (72% of the theoretical (wt. %)).

Second step—N-(6-amino-2,3-dichlorobenzyl) glycine ethyl ester dihydrochloride (MW 350.1)

First, 1048 g of N-(2,3-dichloro-6-nitrobenzyl) glycine ethyl ester hydrochloride were suspended in 11,980 ml of ethanol in a 20-liter reaction flask at room temperature while stirring and then 700 ml of 5-6 N hydrochloric acid (in 2-propanol) were added. Then the entire reaction apparatus was flushed with nitrogen. 104 g of palladium on activated charcoal (10% palladium), which had first been made into a slurry in approximately 300 ml of ethanol, were added to this reaction mixture. After evacuating the reaction apparatus, hydrogen was introduced and the hydrogenation was conducted at 0.3 bar until approximately 97% of the initial material was converted. The reaction mixture was heated during the hydrogenation. The conversion was followed by means of HPLC. Then the still warm reaction mixture was aspirated through a filter preheated to approximately 80° C. The catalyst was washed with approximately 600 ml of ethanol. The liquid phases were combined and cooled to 4-8° C. while stirring. The precipitated dihydrochloride of the final product was aspirated, washed with 300 ml of ethanol, and dried in the drying cabinet at approximately 60° C. The yield of the product N-(2,3-dichloro-6-nitrobenzyl) glycine ethyl ester hydrochloride* amounted to 726 g (68% of the theoretical (wt. %)).
*sic; dihydrochloride?—Trans. Note.

Third step—Ethyl-(2-amino-5,6-dichloro-2H-quinazolin-3-yl) acetate hydrobromide
(final product)

First, 940 g of N-(6-amino-2,3-dichlorobenzyl) glycine ethyl ester dihydrochloride were suspended in 4950 ml of ethanol in a 10-liter reaction flask at room temperature while stirring and then this suspension was cooled to approximately 8° C. Then approximately 1733 ml of sodium ethylate solution (20 wt. % NaOEt) were slowly added and adjusted to a pH between 11.2 and 11.8. The sodium chloride that formed was aspirated until the reaction mixture was clear. 2227 ml of cyanogen bromide solution (c=250 g/L in ethanol) were added while stirring to the clear reaction mixture. The reaction mixture was stirred for approximately 18 h at room temperature after the addition and the conversion to the final product was monitored by means of HPLC. The yield of the final product N-(2,3-dichloro-6-nitrobenzyl) glycine ethyl ester hydrochloride* amounted to 770 g (75% of the theoretical (wt. %)).
*sic; hydrobromide?—Trans. Note.

In order to obtain the final product, the reaction mixture was cooled to approximately 5° C., the precipitated final product was aspirated and washed with ethanol. Then the final product was dried in the drying cabinet.

B) Methyl-(2-amino-5,6dichloro-2H-quinazolin-3-yl) acetate hydrochloride (MW 324.6)

First step—N-(2,3-dichloro-6-nitrobenzyl) glycine methyl ester hydrochloride (MW 329.6) The reaction was conducted as described under A) First step, wherein glycerin methyl ester hydrochloride was utilized instead of glycerin ethyl ester hydrochloride. The yield of crude product amounted to 903 g (73% of the theoretical (wt. %)).

Second step—N-(6-amino-2,3-dichlorobenzyl) glycine methyl ester dihydrochloride (MW 336.1) The reaction was conducted as described under A) Second step, wherein glycerin methyl ester hydrochloride was utilized instead of glycerin ethyl ester hydrochloride. The yield of product amounted to 740 g (62% of the theoretical (wt. %)).

Third step—Methyl-(2-amino-5,6-dichloro-2H-quinazolin-3-yl) acetate hydrochloride
(final product)

The reaction was conducted as described under A) Third step, wherein glycerin methyl ester dihydrochloride was utilized instead of glycerin ethyl ester dihydrochloride. A cyanogen chloride solution (c=145 g/L in ethanol) was used for the conversion, wherein the reaction was conducted in a closed reaction vessel at room temperature.

The yield of crude product amounted to 650 g (75% of the theoretical (wt. %)).

### COMPARATIVE EXAMPLE 1

### Methyl-(2-amino-5,6-dichloro-2H-quinazolin-3-yl) acetate hydrochloride

For the cyclization, 0.3 mmol of the anagrelide prodrug methyl-(2-amino-5,6-dichloro-2H-quinazolin-3-yl) acetate hydrochloride (analysis: UV-Maxima 217,262 nm; MW 324.59; mp>250° C.), corresponding to the general formula I, wherein R1 indicates an alkyl group with one carbon atom, R2 and R3 each indicate a hydrogen atom, and R4 and R5 each indicate a chlorine atom, was added to 10 ml of an aqueous hydrochloric acid solution (0.1 M HCl), at 37° C. with slight stirring. After one hour, 9.6% of the prodrug was converted to anagrelide.

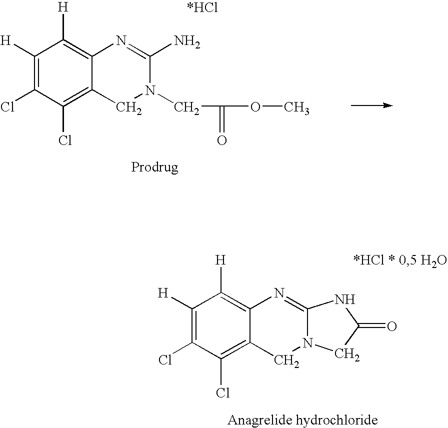

### COMPARATIVE EXAMPLE 2

### Methyl-(2-amino-5,6-dichloro-2H-quinazolin-3-yl) acetate hydrochloride

For the cyclization, 0.3 mmol of the anagrelide prodrug methyl-(2-amino-5,6-dichloro-2H-quinazolin-3-yl) acetate hydrochloride (MW 324.59) corresponding to the general formula I, wherein R1 indicates an alkyl group with one carbon atom, R2 and R3 each indicate a hydrogen atom, and R4 and R5 each indicate a chlorine atom, was added to 10 ml of pH-neutral water, at 37° C. with slight stirring. After one hour, 58% of the prodrug was converted to anagrelide.

### EXAMPLE 1

### Methyl-(2-amino-5,6-dichloro-2H-quinazolin-3-yl) acetate hydrochloride

For the cyclization, 0.3 mmol of the anagrelide prodrug methyl-(2-amino-5,6-dichloro-2H-quinazolin-3-yl) acetate hydrochloride corresponding to the general formula I, wherein R1 indicates an alkyl group with one carbon atom, R2 and R3 each indicate a hydrogen atom, and R4 and R5 each indicate a chlorine atom, was added to 10 ml of an aqueous basic solution (0.1 M NaOH), at 37° C. with slight stirring. After one hour, 100% of the prodrug was converted to anagrelide.

### EXAMPLE 2

### Ethyl-(2-amino-5,6-dichloro-2H-quinazolin-3-yl) acetate hydrochloride hemihydrate

For the cyclization, 0.3 mmol of the anagrelide prodrug ethyl-(2-amino-5,6-dichloro-2H-quinazolin-3-yl) acetate hydrochloride hemihydrate, corresponding to the general formula I, wherein R1 indicates an alkyl group with two carbon atoms, R2 and R3 each indicate a hydrogen atom, and R4 and R5 each indicate a chlorine atom, was added to 10 ml of an aqueous basic solution (15 g of sodium hydrogen carbonate in 1 liter of water), at 37° C. with slight stirring. This basic solution was selected because it corresponds to a common test system for simulation of the milieu of the intestinal juice (pH between 8 and 10). After one hour, 100% of the prodrug was converted to anagrelide.

### EXAMPLE 3

### Methyl-(2-amino-5,6-dichloro-2H-quinazolin-3-yl) acetate hydrochloride hemihydrate

For the cyclization, 0.3 mmol of the anagrelide prodrug methyl-(2-amino-5,6-dichloro-2H-quinazolin-3-yl) acetate hydrochloride hemihydrate, corresponding to the general formula I, wherein R1 indicates an alkyl group with one carbon atom, R2 and R3 each indicate a hydrogen atom, and R4 and R5 each indicate a chlorine atom, was added to 10 ml of an aqueous basic solution (15 g of sodium hydrogen carbonate in 1 liter of water), at 37° C. with slight stirring. This basic solution was selected because it corresponds to a common test system for simulation of the milieu of the intestinal juice (pH between 8 and 10). After one hour, 100% of the prodrug was converted to anagrelide.

### EXAMPLE 4

### Ethyl-(2-amino-5,6-dichloro-2H-quinazolin-3-yl) acetate hydrobromide

For the cyclization, 0.3 mmol of the anagrelide prodrug ethyl-(2-amino-5,6-dichloro-2H-quinazolin-3-yl) acetate hydrobromide (analysis: UV maxima 204, 216, 262 nm; mp 291-293, corresponding to the general formula I, wherein R1 indicates an alkyl group with two carbon atoms, R2 and R3 each indicate a hydrogen atom, and R4 and R5 each indicate a chlorine atom, was added to 10 ml of an aqueous basic solution (15 g of sodium hydrogen carbonate in 1 liter of water), at 37° C. with slight stirring. This basic solution was selected because it corresponds to a common test system for simulation of the milieu of the intestinal juice (pH between 8 and 10). After one hour, 100% of the prodrug was converted to anagrelide.

### EXAMPLE 5

### In Vivo Experiment

### Methyl-(2-amino-5,6-dichloro-3,4-dihydro-2H-quinazolin-3-yl) acetate hydrochloride

In the following example, in contrast to Examples 1-4, the anagrelide prodrug methyl-(2-amino-5,6-dichloro-3,4-dihydro-2H-quinazolin-3-yl) acetate hydrochloride was investigated in different concentrations in an animal experiment (in vivo) on 6 rats relative to the absorption and the course of the blood concentration of the anagrelide formed after a single oral administration.

For this purpose, the prodrug was dissolved in distilled water and orally administered to the experimental animals (each time in a duplicate test) by means of a stomach probe. The pharmacokinetics were determined by sampling blood from the retroorbital venus plexus 1, 2, 4 and 8 hours after the administration of the substance. For this purpose, the concentrations of anagrelide and its decomposition products were determined in the blood specimens by means of HPCL-MS. The results are shown in the following Table 1.

TABLE 1
Concentration of anagrelide and its decomposition
products in the blood of rats, formed from the anagrelide
prodrug methyl-(2-amino-5,6-dichloro-3,4-dihydro-2H-quinazolin-3-yl)
acetate hydrochloride

| | | | | | Anagrelide |
|---|---|---|---|---|---|
| | Experi- | Dose of | | | decomposition |
| Serial | mental | prodrug | | Anagrelide | products |
| No. | animal | [mg/kg] | Time [h] | [ng/ml] | [ng/ml] |
| 1 | 11 | 1 | 1 | 88.7 | <DL |
| 2 | | | 2 | <DL | <DL |
| 3 | | | 4 | <DL | <DL |
| 4 | | | 8 | <DL | <DL |
| 5 | 12 | 1 | 1 | 81.7 | <DL |
| 6 | | | 2 | <DL | <DL |
| 7 | | | 4 | <DL | <DL |
| 8 | | | 8 | <DL | <DL |
| 9 | 21 | 3 | 1 | 684.7 | <DL |
| 10 | | | 2 | 513.6 | <DL |
| 11 | | | 4 | 127.2 | <DL |
| 12 | | | 8 | <DL | <DL |
| 13 | 22 | 3 | 1 | 710.3 | <DL |
| 14 | | | 2 | 483.8 | <DL |
| 15 | | | 4 | 153.5 | <DL |
| 16 | | | 8 | <DL | <DL |
| 17 | 31 | 10 | 1 | 4176.2 | 44.9 |
| 18 | | | 2 | 4051.0 | <DL |
| 19 | | | 4 | 1828.2 | <DL |
| 20 | | | 8 | 514.4 | <DL |
| 21 | 32 | 10 | 1 | 6079.1 | 41.2 |
| 22 | | | 2 | 5859.2 | <DL |
| 23 | | | 4 | 4023.6 | 53.5 |
| 24 | | | 8 | 1918.3 | 61.3 |

<DL - value lay below the detection limit of 40 ng/ml.

The results show that anagrelide is formed rapidly in the body of the animal after administration as a function of the concentration of the prodrug that is utilized, and then can be detected in the blood of the animal. The concentration curves of the anagrelide formed and the formation of typical decomposition products show that the anagrelide formed is also metabolized. In the case of higher initial concentrations of the prodrug, typical decomposition products of the anagrelide can also be detected. The appearance of anagrelide and anagrelide decomposition products in the blood shows that the anagrelide prodrug is suitable for utilization as a precursor of the anagrelide. The in vivo experiments thus support the results of Examples 1-4.

All of the disclosed features as well as combinations of the disclosed features are the subject of the invention, insofar as these are not expressly designated as known. Of course, the preceding Description and the preceding Examples are given only by way of illustration and are not limiting. Many embodiments can be recognized by the person skilled in the art when he examines the preceding Description and the Examples. The scope of protection for the invention thus should not be established relative to the preceding Description and the preceding Examples, but rather it should be established relative to the following patent claims, together with the full scope of protection for equivalents which these patent claims also cover. Insofar as papers, scientific articles, patents and patent applications are named in this application, their disclosure is expressly included herewith in the present Description.

1. Use of 2-amino-2H-quinazoline derivatives of the general chemical formula I 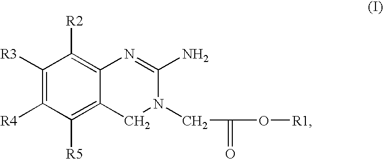
wherein R1 is an alkyl group with 1-5 carbon atoms and R2, R3, R4 and R5, independently of one another, each indicate a chlorine or hydrogen atom, as well as their pharmaceutically compatible salts, for producing therapeutic agents for the treatment of myeloproliferative diseases and for bronchodilation in mammals.

2. The use according to claim 1, further characterized in that at least two residues of R2, R3, R4 and R5 each indicate chlorine atoms.

3. The use according to claim 1, further characterized in that R1 stands for methyl or ethyl, R2 and R3 are each hydrogens, and R4 and R5 are each chlorine atoms.

4. The use according to claim 1, further characterized in that the pharmaceutically compatible salts are selected from the group comprising the hydrochloride, hydrobromide, sulfate, fumarate, maleate, lactate and succinate.

5. The use according to claim 1, further characterized in that the 2-amino-2H-quinazoline derivatives are selected from the group comprising methyl-(2-amino-5,6-dichloro-3,4-dihydro-2H-quinazolin-3-yl) acetate hydrochloride hemihydrate, methyl-(2-amino-5,6-dichloro-3,4-dihydro-2H-quinazolin-3-yl) acetate hydrobromide hemihydrate, ethyl-(2-amino-5,6-dichloro-3,4-dihydro-2H-quinazolin-3-yl) acetate hydrochloride hemihydrate, and ethyl-(2-amino-5,6-dichloro-3,4-dihydro-2H-quinazolin-3-yl) acetate hydrobromide hemihydrate.

6. Use of 2-amino-2H-quinazoline derivatives of the general chemical formula I 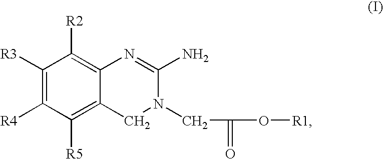
wherein R1 indicates an alkyl group with 1-5 carbon atoms and R2, R3, R4 and R5, independently of one another, each indicate a chlorine or hydrogen atom, and their pharmaceutically compatible salts, for the treatment of myeloproliferative diseases and for bronchodilation in mammals.

7. Use of a pharmaceutical preparation containing at least one agent, selected from the group comprising 2-amino-2H-quinazoline derivatives of the general chemical formula I 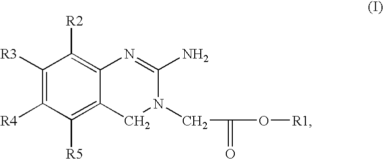
wherein R1 is an alkyl group with 1-5 carbon atoms and R2, R3, R4 and R5, independently of one another, each indicate a chlorine or hydrogen atom, and their pharmaceutically compatible salts, and at least one pharmaceutically compatible adjuvant, for the treatment of myeloproliferative diseases and for bronchodilation in mammals.

8. The use of the pharmaceutical preparation according to claim 7, further characterized in that at least one of the pharmaceutically compatible adjuvants reacts basically in aqueous medium.
